(19) United States
(12) Patent Application Publication
(10) Pub. No.: US 2026/0259344 A1
(43) Pub. Date: Sep. 3, 2026

(54) TECHNIQUES FOR PREDICTING CO2 SATURATION IN FORMATIONS BASED ON NUCLEAR MAGNETIC RESONANCE DATA

(71) Applicant: Schlumberger Technology Corporation, Sugar Land, TX (US)

(72) Inventor: Robert Laronga, Houston, TX (US)

(21) Appl. No.: 19/552,674

(22) Filed: Feb. 27, 2026

Related U.S. Application Data
(60) Provisional application No. 63/764,694, filed on Feb. 28, 2025.

(51) Int. Cl. G01V 3/32; E21B 25/00; E21B 43/16; E21B 49/00; G01N 33/24; G01R 33/50; G01V 3/38

(52) U.S. Cl. CPC G01V 3/32; E21B 25/00; E21B 43/164; E21B 49/00; G01N 33/24; G01R 33/50; G01V 3/38

(57) ABSTRACT

Systems and methods for applying a cutoff or gaussian distribution, however measured, derived, or conceived, in the T1 domain, in the T2 domain, or in the two-dimensional T1-T2 domain to a Nuclear Magnetic Resonance (NMR) log acquired in a well, thereby predicting a fraction of a fluid that will be replaced by carbon dioxide (CO₂) in response to the CO₂ being introduced into a reservoir pore system (e.g., the pore system of reservoir rock of a geological formation and the fluid it contains).

### CROSS-REFERENCE TO RELATED APPLICATION

This application claims priority to, and the benefit of, U.S. Provisional Patent Application Ser. No. 63/764,694, entitled “TECHNIQUES FOR PREDICTING CO2 SATURATION IN FORMATIONS BASED ON NUCLEAR MAGNETIC RESONANCE DATA”, which was filed on Feb. 28, 2025, and which is herein incorporated by reference in its entirety for all purposes.

### BACKGROUND

The present disclosure generally relates to techniques for predicting carbon dioxide (CO₂) saturation in reservoir rocks and/or sealing formations based on nuclear magnetic resonance (NMR) data indicative of pore size information.

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as an admission of any kind.

As part of the global effort to achieve net-zero by 2050, various Carbon Capture and Sequestration (CCS) projects, Carbon Capture, Usage, and Storage (CCUS) projects, and Enhanced Oil Recovery (EOR) projects aim to store large amounts of CO₂ in underground formations. While many types of target formations may be considered for storage, each with its own specific technical challenges, each project has the following risks in common: (1) ensuring that the envisioned quantity of CO₂ can be safely and economically stored over the intended operating phase of the project, and (2) ensuring that CO₂ remains contained in the subsurface with permanence and within a designated zone.

### SUMMARY

A summary of certain embodiments described herein is set forth below. It should be understood that these aspects are presented merely to provide the reader with a brief summary of these certain embodiments and that these aspects are not intended to limit the scope of this disclosure.

Various embodiments of the present disclosure are directed to applying a cutoff or gaussian distribution, however measured, derived, or conceived, in the T1 domain, in the T2 domain, or in the two-dimensional T1-T2 domain to a Nuclear Magnetic Resonance (NMR) log acquired in a well, thereby predicting a fraction of a fluid that will be replaced by carbon dioxide (CO₂) in response to the CO₂ being introduced into a reservoir pore system (e.g., the pore system of reservoir rock of a geological formation and the fluid it contains).

Initially, the fluids contained in the pore system will normally include water and/or brine. Introducing CO₂ to the pore system via an injection well means applying pressure in excess of the reservoir pressure to force CO₂ into the pore space, displacing some of the native brine back into the reservoir. As such, the embodiments described herein should not be confused with fluids in a wellbore, or with fluids used to drill the wellbore. Rather, the embodiments described herein include novel methods to predict the percentage of water/brine that will be displaced by injected CO₂ in the reservoir pore space.

Various refinements of the features noted above may be undertaken in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings in which:

FIG. 1 is a partial cross-sectional view of a nuclear magnetic resonance (NMR) tool that may be used to receive and/or analyze NMR data in a reservoir pore system, in accordance with embodiments of the present disclosure;

FIG. 2 depicts a data processing system configured to control the reservoir pore system of FIG. 1, in accordance with embodiments of the present disclosure;

FIG. 3 illustrates still images from an animation of a spin echo, in accordance with embodiments of the present disclosure;

FIG. 4 illustrates a single Carr-Purcell-Meiboom-Gill (CPMG) echo train, in accordance with embodiments of the present disclosure;

FIG. 5 is a diagram of a typical T1-T2 measurement pulse sequence that consists of multiple CPMGs, in accordance with embodiments of the present disclosure;

FIG. 6 is a diagram of an NMR signal observed in a typical CPMG of a T1-T2 measurement sequence, in accordance with embodiments of the present disclosure;

FIG. 7 illustrates a method for combining NMR log data with core sample analysis to determine various parameters relating to CO₂ to be injected into a reservoir pore system, in accordance with embodiments of the present disclosure;

FIG. 8 illustrates another method for combining NMR log data with core sample analysis to determine various parameters relating to CO₂ to be injected into a reservoir pore system, in accordance with embodiments of the present disclosure;

FIG. 9 illustrates another method for combining NMR log data with core sample analysis to determine various parameters relating to CO₂ to be injected into a reservoir pore system, in accordance with embodiments of the present disclosure; and

FIG. 10 illustrates another method for combining NMR log data with core sample analysis to determine various parameters relating to CO₂ to be injected into a reservoir pore system, in accordance with embodiments of the present disclosure.

### DETAILED DESCRIPTION OF SPECIFIC EMBODIMENTS

One or more specific embodiments of the present disclosure will be described below. These described embodiments are only examples of the presently disclosed techniques. Additionally, in an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

As used herein, the terms “connect,” “connection,” “connected,” “in connection with,” and “connecting” are used to mean “in direct connection with” or “in connection with via one or more elements”; and the term “set” is used to mean “one element” or “more than one element.” Further, the terms “couple,” “coupling,” “coupled,” “coupled together,” and “coupled with” are used to mean “directly coupled together” or “coupled together via one or more elements.” As used herein, the terms “up” and “down,” “uphole” and “downhole”, “upper” and “lower,” “top” and “bottom,” and other like terms indicating relative positions to a given point or element are utilized to more clearly describe some elements. Commonly, these terms relate to a reference point as the surface from which well operations are initiated as being the top (e.g., uphole or upper) point and the total depth along the axis being the lowest (e.g., downhole or lower) point, whether the well (e.g., wellbore, borehole) is vertical, horizontal or slanted relative to the surface.

In addition, as used herein, the terms “real time”, “real-time”, or “substantially real time” may be used interchangeably and are intended to describe operations (e.g., computing operations) that are performed without any human-perceivable interruption between operations. For example, as used herein, data relating to the systems described herein may be collected, transmitted, and/or used in control computations in “substantially real time” such that data readings, data transfers, and/or data processing steps occur once every second, once every 0.1 second, once every 0.01 second, or even more frequent, during operations of the systems (e.g., while the systems are operating). In addition, as used herein, the terms “automatic” and “automated” are intended to describe operations that are performed are caused to be performed, for example, by a data processing system (i.e., solely by the data processing system, without human intervention).

As mentioned above, various Carbon Capture and Sequestration (CCS) projects, Carbon Capture, Usage, and Storage (CCUS) projects, and Enhanced Oil Recovery (EOR) projects aim to store large amounts of carbon dioxide (CO₂) in underground formations as part of the global effort to achieve net-zero by 2050. While many types of target formations may be considered for storage, each with its own specific technical challenges, each project has the following risks in common: (1) ensuring that the envisioned quantity of CO₂ can be safely and economically stored over the intended operating phase of the project, and (2) ensuring that CO₂ remains contained in the subsurface with permanence and within a designated zone. De-risking such challenges is the object of a reservoir simulation taking place within a three-dimensional framework based on the best possible knowledge of subsurface geology. Simulated phenomena include, at a minimum, two-phase or multiphase hydraulic flow through porous media and accompanying pore pressure changes to predict changes to the distribution of fluids and pressure over time. In certain cases, geomechanical, geochemical, and thermal phenomena may also be simulated.

Hydraulic flow simulations are sensitive to several key reservoir properties pre-populated in the cells of the simulation grid. Such properties include, but are not limited to porosity, effective porosity, initial pore pressure, intrinsic permeability, relative permeability, entry pressure, and capillary pressure profile. While the first four properties can be quantified early in a project by drilling, coring, and logging a stratigraphic test well, the remaining three are not so simple to quantify, as they require the presence of two fluid phases but no CO₂ is typically introduced to the reservoir at the time of evaluation. Closely related to these properties are CO₂ storage efficiency, CO₂ saturation, and CO₂ residual trapping.

CO₂ storage efficiency is the equivalent percentage of pore volume that would be occupied by stored CO₂ if it were all in a free (usually supercritical) phase. CO₂ saturation is the percentage of pore volume occupied by free phase CO₂. For projects where most of the CO₂ is stored in a free phase, it may be convenient to estimate CO₂ storage efficiency as the maximum achievable CO₂ saturation. Storage efficiency may be defined at pore scale (“displacement efficiency”) or at reservoir scale (“volumetric efficiency”). As the flow path of CO₂ typically does not contact the entire considered reservoir, overall storage efficiency is typically an order of magnitude lower when viewed at this scale.

The impact of considering wrong storage efficiency can have serious implications for the accuracy of flow simulation and its value as a risk-management tool. For example, considering efficiency that is higher than reality will typically result in underestimation of the true areal extent of the CO₂ plume at a given time. In reality, the plume might exceed the boundaries of the licensed area, arrive at a legacy wellbore, or interact with a major fault in a manner that was unforeseen and unplanned.

CO₂ residual trapping is the percentage of pore volume that remains saturated with CO₂ after the CO₂ plume has had the opportunity to migrate away, either due to buoyant forces or due to hydrodynamic flow. This CO₂ is held in place by capillary pressure hysteresis; essentially it found a path to enter the pore, displacing brine, but it is unable to find a path to exit the pore.

Where CO₂ is not immediately held in place by a structural trap, residual trapping is an important parameter to predict how much of the CO₂ will migrate, and how far it will go. Where CO₂ is injected at a distance below a confining zone, residual trapping is important to predict how quickly a column of CO₂ will accumulate, and how rapidly overpressure will increase at the base of the confining zone.

Quantifying these properties is typically the domain of laboratory special core analysis (SCAL) techniques that are applied to a limited number of discrete core samples, which are taken to represent the reservoir at large. Practical limitations in time, cost, and available laboratory bandwidth typically mean that only a few samples will be run at most, and some risk will be taken in populating the measured properties as representative of the entire reservoir. Digital SCAL is one existing method by which we can reduce risk, by constructing a calibrated digital twin of the SCAL experiment and then running the digital SCAL model on several samples having a range of properties. However, it is limited to a very small sample size.

Wireline logs provide a continuous vertical profile of reservoir properties at a scale that is larger than SCAL, but still fine in resolution compared to the reservoir simulation. Methods of interpreting mineral composition, true and effective porosity, and estimated intrinsic permeability are well-accepted, and are even more robust when calibrated with discrete data points from Routine Core Analysis (RCAL). In the oil and gas industry, the use of logs to estimate fluid saturations is extremely well-established, recognizing that measuring a column of hydrocarbon that already exists in the subsurface, and has had millions of years to reach a state of equilibrium. In the case of CO₂ storage project evaluation, the formation is normally at 0% CO₂ saturation when the logs are run, thus none of the measurements are representative of the state of saturation we would like to predict.

Nuclear Magnetic Resonance (NMR) measurements are of special interest to the challenge. In water-saturated rocks, the data is inherently sensitive to the surface-to-volume ratio (S/V) of the pores. In the oil and gas industry, simple linear or nonlinear relationships have been assumed between pore size and pore-throat size, a primary factor that determines the capillary pressure profile. This has enabled estimation of capillary pressure distributions directly from the NMR measurements, although such distributions are suitable for systems of water only, oil and water, or methane and water. A different conversion would be required for a system of CO2 and brine. Depending on the rock type, recent literature suggests that rock-specific nonlinear conversions will be required.

NMR measurements are further of interest because as applied to subsurface exploration, they are uniquely sensitive to hydrogen, and no other element. NMR measurements quantify the porosity of a rock sample in proportion to the amount of water (and liquid hydrocarbon) that is present. NMR does not respond at all to CO₂. Thus, when CO₂ substitutes water in the pore system, the result will be a quantifiable decrease in the NMR signal (apparent porosity). By combining a lab NMR spectrometer with core flooding equipment, changes in saturation can be measured at various points during the drainage and imbibition phases of a two-phase flow experiment with CO₂ as the intruding phase in a brine saturated core. Thereafter, T2 distributions before and after introduction of CO₂ into the pore system can be compared to understand which pores the CO₂ can access at a given injection pressure, and which pores remain water saturated.

Finally, NMR measurements are ideal for the challenge at hand because highly equivalent measurements can be made on the benchtop in the laboratory using rock samples as well as downhole in an exploration well in open hole measuring the formation in situ. Laboratory spectrometers operating at a frequency of 2 MHz are commonly available; such spectrometers can be programmed with pulse sequence parameters identical to a 2 MHz downhole electric wireline tool operated by SLB. By drawing a cutoff in the T2 domain, or by representing substituted brine as a gaussian distribution or a non-gaussian distribution (“factor”) based on benchtop measurements, various embodiments of the present disclosure apply these to the continuous logs over the entire interval. Such embodiments can make predictions, depth by depth, of maximum CO₂ saturation, residual trapped CO₂ saturation, CO₂-brine capillary pressure, CO₂ entry pressure, and more. It is noted that the shape that is a component of a histogram (e.g., T2 distribution) is gaussian in the idealized model. In real datasets, the shape may be non-gaussian or asymmetric. This shape is typically called a “factor”. Examples of application of such factors are described in Jain, V., Cao Minh, C., Heaton, N., Ferraris, P., Ortenzi, L., & Ribeiro, M. T. (2013 June). Characterization of Underlying Pore and Fluid Structure Using Factor Analysis on NMR Data. Paper presented at the SPWLA 54th Annual Logging Symposium, New Orleans, Louisiana. Paper Number: SPWLA-2013-TT, the general techniques within which are incorporated by reference herein.

In general, in a reservoir pore system, you expect to find pores that are almost entirely saturated with water. A main question may be how much CO₂ you are going to be able to store in the pore space of a particular well. One main factor is the ability of the CO₂ to push the water or brine into the reservoir, such that the CO₂ may be sequestered. In certain embodiments, some CO₂ may be injected into the rock and NMR measurements may be taken before and after to determine the effect of the CO₂ injection (e.g., either in a lab or in the well). In general, a goal is to figure out just how much of a percentage of CO₂ injection will occur for any particular pore system.

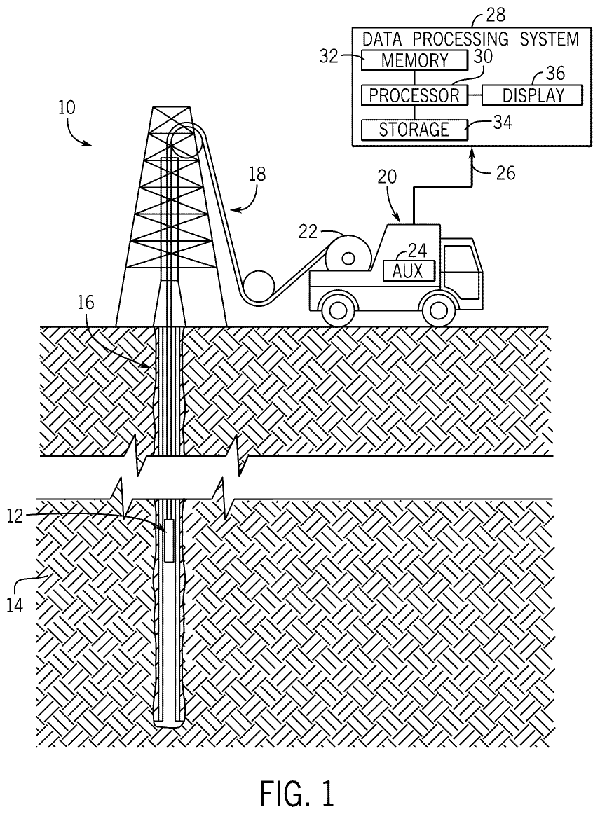

With this in mind, FIG. 1 illustrates a reservoir pore system 10 that may employ the systems and methods of this disclosure. The reservoir pore system 10 may be used to convey an NMR tool 12 through a geological formation 14 via a wellbore 16. The NMR tool 12 may be conveyed on a cable 18 via a logging winch system 20. Although the logging winch system 20 is schematically shown in FIG. 1 as a mobile logging winch system carried by a truck, the logging winch system 20 may be substantially fixed (e.g., a long-term installation that is substantially permanent or modular). Any suitable cable 18 for well logging may be used. The cable 18 may be spooled and unspooled on a drum 22 and an auxiliary power source 24 may provide energy to the logging winch system 20 and/or the NMR tool 12. As used herein, the term “reservoir pore system” is used to mean the pore system of reservoir rock of a geological formation 14 and the fluid it contains.

Moreover, although the NMR tool 12 is described herein as a wireline downhole tool, it should be appreciated that any suitable conveyance may be used. For example, the NMR tool 12 may instead be conveyed as a logging-while-drilling (LWD) tool as part of a bottom hole assembly (BHA) of a drill string, conveyed on a slickline or via coiled tubing, and so forth. For the purposes of this disclosure, the NMR tool 12 may be any suitable measurement tool that obtains NMR logging measurements through depths of the wellbore 16.

Many types of downhole tools may obtain NMR logging measurements in the wellbore 16. These include, for example, nuclear magnetic resonance (NMR) tools such as the Combinable Magnetic Resonance (CMR) tool, the Magnetic Resonance Scanner (MRX) tool, and the ProVISION tool by Schlumberger Technology Corporation. In general, NMR tools may have a permanent magnet that produces a static magnetic field at a desired test location (e.g., where the fluid is located). The static magnetic field produces an equilibrium magnetization in the fluid that is aligned with a magnetization vector along the direction of the static magnetic field. A transmitter antenna produces a time-dependent radio frequency magnetic field that is perpendicular to the direction of the static field. The radio frequency magnetic field produces a torque on the magnetization vector that causes it to rotate about the axis of the applied radio frequency magnetic field. The rotation results in the magnetization vector developing a component perpendicular to the direction of the static magnetic field. This causes the magnetization vector to align with the component perpendicular to the direction of the static magnetic field, and to process around the static field.

The time for the magnetization vector to re-align with the static magnetic field is known as the longitudinal magnetization recovery time, or “T1 relaxation time.” The spins of adjacent atoms process in tandem synchronization with one another due to the precession of the magnetization vector. The time for the precession of the spins of adjacent atoms to break synchronization is known as the transverse magnetization decay time, or “T2 relaxation time.” Thus, the measurements obtained by the NMR tool 12 may include distributions of the first relaxation time T1, the second relaxation time T2, or molecular diffusion D, or a combination of these. For example, a downhole NMR tool 12 may measure just T2 distribution, or the tool may measure a joint T1-T2 distribution or T1-T2-D distribution.

For each depth of the wellbore 16 that is measured, a downhole NMR tool may generate NMR logging measurements that include a distribution of amplitudes of T2 relaxation time, T1 relaxation time, diffusion, or a combination thereof. This list is intended to present certain examples and is not intended to be exhaustive. Indeed, any suitable NMR tool 12 that obtains NMR logging measurements may benefit from the systems and methods of this disclosure.

The NMR tool 12 may provide NMR logging measurements 26 to a data processing system 28 via any suitable telemetry (e.g., via electrical signals pulsed through the geological formation 14 or via mud pulse telemetry). The data processing system 28 may process the NMR logging measurements 26 to identify patterns in the NMR logging measurements 26. The patterns in the NMR logging measurements 26 may indicate certain properties of the wellbore 16 (e.g., viscosity, porosity, permeability, relative proportions of water and hydrocarbons, and so forth) that might otherwise be indiscernible by a human operator.

To this end, the data processing system 28 thus may be any electronic data processing system that can be used to carry out the systems and methods of this disclosure. For example, the data processing system 28 may include a processor 30, which may execute instructions stored in memory 32 and/or storage 34. As such, the memory 32 and/or the storage 34 of the data processing system 28 may be any suitable article of manufacture that can store the instructions. The memory 32 and/or the storage 34 may be ROM memory, random-access memory (RAM), flash memory, an optical storage medium, or a hard disk drive, to name a few examples. A display 36, which may be any suitable electronic display, may provide a visualization, a well log, or other indication of properties in the geological formation 14 or the wellbore 16 using the NMR logging measurements 26.

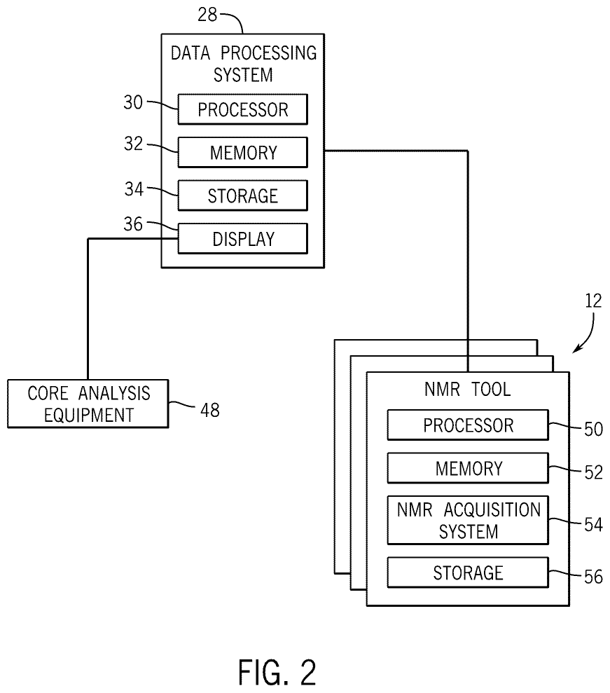

Operation of the reservoir pore system 10 may be controlled by a processor of the data processing system 38. For example, FIG. 2 illustrates a block diagram of the data processing system 38 that is communicatively coupled to the NMR tool 12. In the illustrated embodiment, the NMR tool 12 may include a processor 50, memory 52, an NMR acquisition system 54, and storage 56. It should be noted that, in certain embodiments, the NMR tool 12 may not include the memory 52 or the storage 56 but, rather, data acquired by the NMR tool 12 may be communicated to the data processing system 28 via the wireline conveyance. In some embodiments, the processor 50 may be ASIC (application specific integrated circuit), field programmable gate array (FPGA), a micro control unit (MCU), a digital signal processor (DSP), and the like. In general, the NMR tool 12 communicates with the data processing system 38 via a data cable, telemeter or other suitable techniques. For example, the NMR tool 12 may communicate NMR measurements obtained by an NMR sensor of the NMR tool 12. In turn, a processor of the data processing system 38 may determine certain parameters (e.g., T1, T2, a porosity, a T1/T2 ratio, a water saturation, a permeability) based on the NMR measurements. In such embodiments, the NMR acquisition system 54 may include an emission source (e.g., an antenna) to acquire, obtain, or otherwise measure NMR measurements.

In general, the data processing system 38 may be configured to control analysis procedures of a core sample performed by core analysis equipment 48, as described in greater detail herein. For example, based on the core analysis, the various types of offsets, gaussian distributions, and so forth, described in greater detail herein may be derived by the data processing system 38 and applied to NMR log data acquired by the NMR tool 12 as also described in greater detail herein.

### Multi Wait Time NMR Measurements and T1-T2 Inversion

The main building block of NMR applications for petrophysics both for in-situ logging and laboratory measurements is CPMG (i.e., Carr-Purcell-Meiboom-Gill sequence). To produce CPMG, a static magnetic field B₀ is initially applied for a specific time to the object of interest to polarize the protons or other magnetic nuclei (i.e., to align their magnetic spins in the direction of the static magnetic field). Complete polarization means that all spin vectors are oriented in the direction of the static magnetic field. It corresponds to the maximum object magnetization M₀. With time, actual magnetization M approaches the maximum M₀ exponentially. Characteristic time of that exponent is called T1—longitudinal or spin-lattice relaxation time. Thus, depending on the time of the static magnetic field application (i.e., wait time), the polarization can be practically complete or partial.

In the static magnetic field, the spins experience precession around the magnetic field vector with the certain frequency f=γB₀/2π that is called the Larmor frequency. Here γ is the gyromagnetic ratio, which is a measure of the strength of the nuclear magnetism. Subsequently, tipping magnetic field B₁, oscillating with the Larmor frequency and perpendicular to B₀, is applied for a certain time t to reorient the spins. A corresponding pulse of an oscillating electro-magnetic field is called a radio-frequency (RF) pulse since the Larmor frequency for typical NMR devices lies in the radio frequencies range. The tipping angle is the angle of the spins' reorientation—θ=γB₁τ. The first RF pulse in CPMG has a nominal tipping angle of 90°. It reorients the magnetization from the direction longitudinal to the static field B₀ to a transverse plane. After an initial RF pulse is finished, spins start to experience their precession being contained in that transverse plane and dephasing starts. Microscopic magnetic field inhomogeneities result in some spins precession being slower due to local filed strength and some being faster. Slower precessing spins start to progressively trail behind while faster start getting ahead. Thus, spins start to lose phase coherency. As dephasing progresses, the net magnetization decreases exponentially with time. Characteristic time of that exponent is called T2-transverse or spin-spin relaxation time.

During this dephasing process, observations of spin echoes are made. The proton spins in the transverse plane are re-phased with the application of 180° tipping angle RF pulse. If a transverse magnetization vector has phase angle α, then application of a 180° oscillating field pulse changes the phase angle to −α. The phase order of the spins is therefore reversed, and the slower precessing spins are put ahead of the faster ones. The faster spins overtake the slower ones, rephasing occurs, and the receiver antenna coil detects a signal. This signal is called a spin echo. If time t transpires between the application of the 90° RF pulse and the 180° RF pulse, then the same time t will transpire between the application of the 180° RF pulse and the peak of the spin echo. This time between 90° RF pulse and the spin echo that is twice the time between 90° DF pulse and 180° RF pulse is called the echo time.

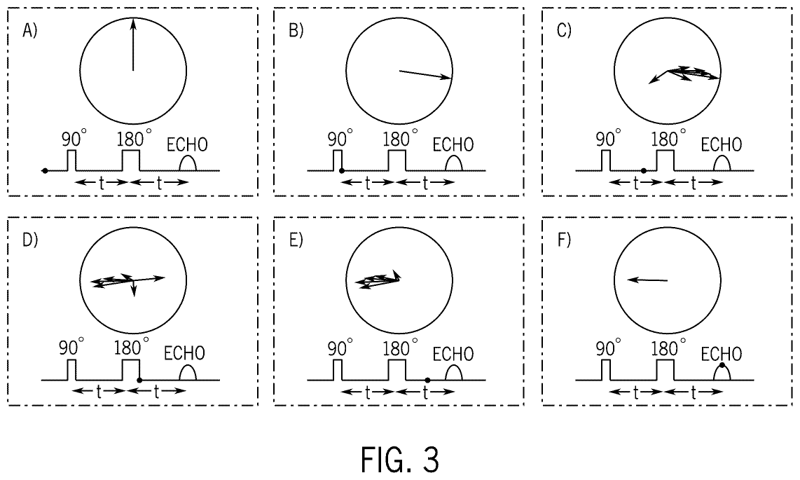

FIG. 3 illustrates still images from an animation of a spin echo by Gavin W Morley, CC BY-SA 3.0<https://creativecommons.org/licenses/by-sa/3.0>, via Wikimedia Commons. The red arrows may be thought of as spins. Initially, they are polarized parallel to the static magnetic field (A). Applying the first pulse (B) rotates the spins by 90° positioning them in the transverse plane. The spins then “spread out”, experiencing decoherence (C) because each is in a slightly different environment. This decoherence is refocused by a second pulse that rotates the spins by 180° (D). Phase order reverses and slower spins “catch up” with faster spins (E). Finally, refocusing occurs and spin echo is recorded (F).

As soon as decoherence progresses in the transverse plane, the share of spins that are re-focused by 180° RF pulse decreases. Thus, repetition of 180° pulses after a single 90° pulse with some certain constant time spacing (e.g., Carr-Purcell time) and recording maximum amplitude of the corresponding echoes is used to measure the dephasing process. The, corresponding procedure of waiting for a certain polarization in a static magnetic field B₀, then applying one 90° tipping angle RF pulse and then a series of evenly spaced in time 180° RF pulses combined with recording of the spin echo amplitudes vs. time in the receiver antenna coil is known as a CPMG sequence. The CPMG sequence renders the echo train (e.g., that is an evenly spaced time series of spin echoes amplitudes). The time spacing of the echo train is called echo spacing TE. Its value is twice the Carr-Purcell time (e.g., the time spacing between 180° RF pulses).

After a period of dephasing equal to several times of the maximum T2, this dephasing is essentially complete, and further rephasing renders no spin echo. The first 90° RF pulse of the CPMG sequence reorients the polarization, eliminating any existing longitudinal polarization while subsequent 180° RF pulses suppress the buildup of new longitudinal polarization. Hence, the spins are completely randomized at the end of a CPMG sequence. To start the next CPMG sequence, the spins must be polarized again. So, a wait time during which repolarization occurs is necessary between the end of one CPMG sequence to the start of the next.

The spin echo signal from the receiver antenna coil is fed into a phase-sensitive detector that outputs two channels of data 90° apart (that phase is not to be confused with 90° tipping angle of the pulse). From these two sensors spin echo apparent signal amplitude and phase can be computed. The phase of the spin echo signal for different time instances in the CPMG sequence and even for different CPMG sequences in one NMR observation is expected to be constant. Therefore, phase variations are attributed to the measurement noise. From these variations, the noise can be estimated together with the phase. Certain phase angle of ¢ is assumed and then ¢ phase correction applied to the data from the both channels with the phase 90° apart. This phase correction is equivalent to rotating the data of these two channels through an angle of ¢. After rotation, one channel will contain primarily the NMR signal while the other channel will contain primarily noise. The value of the proper ¢ is determined as one that minimized noise. Together with the phase angle, the baseline correction or offset (i.e., the value of the detector measurements corresponding to the zero magnetization) is determined in similar manner as one that minimizes value attributed to noise after application of the baseline and phase correction. The noise value determined from the corrected CPMG data may be used in the inversion, as described in greater detail below. The embodiments described herein do not involve modification of the phase rotation and baseline correction algorithms, and assumes that the algorithms established in the industry are used.

The spin echo signal and noise data extracted from the acquisition device are proportional to the magnetization producing the echoes but dependent on the device properties. However, the goal of the measurement is to determine device independent quantities, namely volume shares of different fluids containing magnetic nuclei with their respective T1 and T2 relaxation times. For this purpose, antenna gain correction, temperature correction, and calibration may be performed. Antenna gain correction makes the data independent on the receiver coil antenna properties. Then, temperature correction is done to make the data proportional to the concentration of magnetic nuclei. This can be done because maximum magnetization M₀ for the specific magnetic nuclei is proportional to the product of their concentration to the magnitude of the static magnetic field B₀ divided by absolute temperature of the object of interest. Since in typical NMR measurement (oil/gas and medical) magnetic nuclei are the same, namely protons-hydrogen nuclei, antenna gain and temperature correction make the signal amplitude proportional to the product of proton concentration and B₀². Since B₀ is a known device property, the proportionality coefficient may be determined by calibration—i.e., by performing NMR measurement for a fluid with hydrogen magnetic nuclei of known volume share, T1 and T2 and then determining proper calibration coefficient to achieve that known porosity. This fluid is typically doped water so the known volume share is 1. Doping is done to reduce T1 and T2 that reduces necessary acquisition time and makes calibration easier and more reliable. The embodiments described herein do not involve modification of the antenna gain correction, temperature correction, and calibration algorithms, and assumes that the algorithms established in the industry are used.

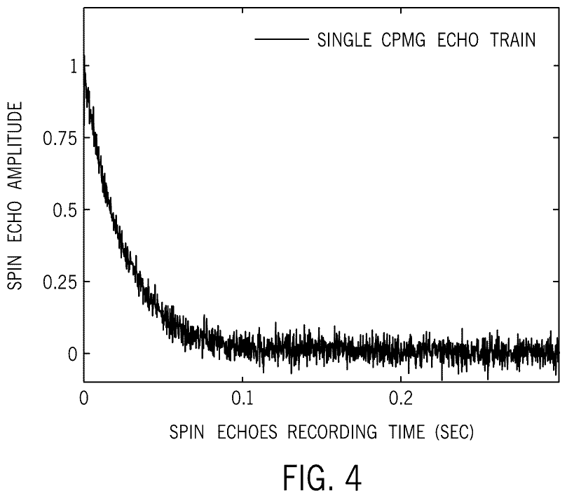

FIG. 4 illustrates a single CPMG echo train—i.e., phase rotated, antenna gain and temperature corrected calibrated spin echo amplitude value vs. time. CPMG is recorded in a laboratory for a doped water with 2.0 sec wait time and contains 1800 echoes with spacing time TE=0.2 msec. The doped water is expected to have T1≈T2≈25 msec. Assuming full polarization before the 90° RF pulse and the absence of noise the n-th spin echo amplitude (e.g., that from now on is assumed to be phase rotated, antenna gain and temperature corrected calibrated spin echo amplitude at the moment of time t=n·TE) for a fluid of relative volume P with single T2 relaxation time is given by the equation:

$${E}_{n} = E ( t = n \cdot TE ) = P \cdot exp ( - \frac{n \cdot TE}{T 2} ) \qquad ( 1 )$$

Equation (1) assumes that the applied magnetic field is uniform and static (i.e., no time variation) with respect to the sample. Moreover, an NMR sensor coil (e.g., antenna) circuit is assumed to be at resonance with Larmor frequency with zero bandwidth (i.e., antenna Q is assumed to be infinite). Noise wₙ is expressed as an additive term to the equation. As a rule, it is considered to be zero mean Gaussian independent on spin echo index n.

$${E}_{n} = P \cdot exp ( - \frac{n \cdot TE}{T 2} ) + {w}_{n} \qquad ( 2 )$$

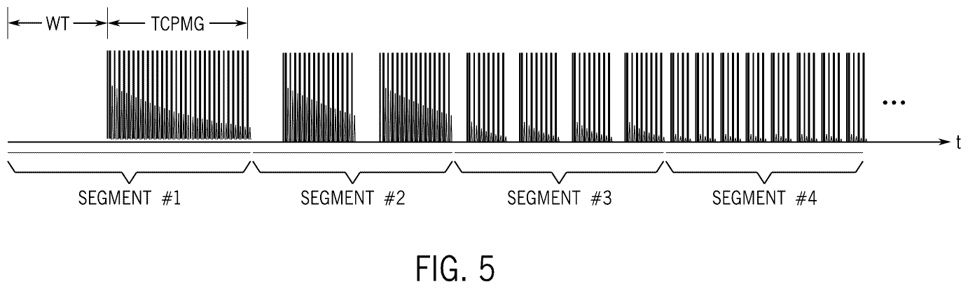

FIG. 5 is a diagram of a typical T1-T2 measurement pulse sequence that consists of multiple CPMGs. In order to characterize both T1 and T2 relaxation times of the fluids under investigation, the NMR acquisition sequence is designed to have multiple CPMGs separated by wait time (WT) periods. During a WT period, there are no RF pulses and spin polarization governed by T1 occurs. Such sequence is called a T1-T2 sequence. FIG. 5 shows a typical T1-T2 sequence.

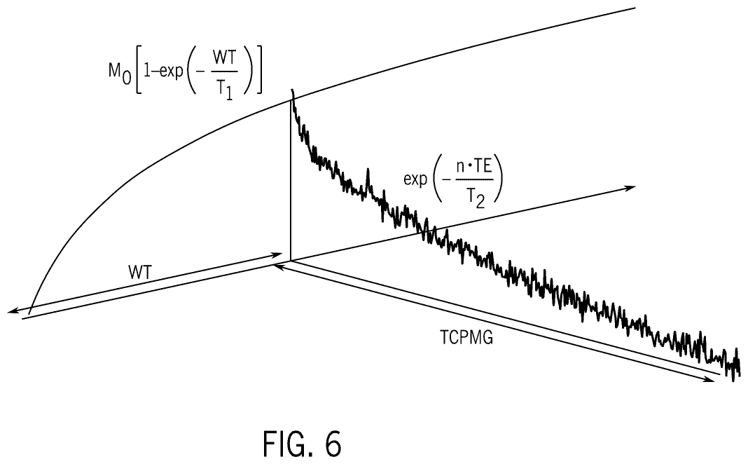

FIG. 6 is a diagram of an NMR signal observed in a typical CPMG of a T1-T2 measurement sequence. It consists of a series of CPMG sequences with different WTs, numbers of echoes (NEs), and TEs to cover to a wide range of samples. Each of these sequences involves polarization during a WT period followed by spin echoes observation as shown in FIG. 6. The long echo train with long WTs has sensitivity to long T1-T2 components, but it is often executed only once or a few times due to long execution time. On the other hand, short echo trains with short WTs are often executed multiple times to ensure enough sensitivity to short T1 and short T2. In addition, short echo trains often use short TEs to capture short T2 components that decay quickly.

Moreover, CPMG sets are normally collected in pairs. After acquisition of the first set, the phase of the RF pulses is changed by 180° (not to be confused with the 180° tipping angle) for the second set acquisition. That renders negative spin echoes amplitudes of the same magnitude. The second set is then subtracted from the first set to produce a phase-alternate pair (PAP). This procedure called phase-alternate pulse sequence (PAPS) technique is designed to preserve the signal and eliminate low-frequency electronic offsets.

Series of CPMG sets with the same WT, NE, and TE is called sub-measurement. Assuming that k is the sub-measurement index with a number of sub-measurements being designated as NS, l is the index of CPMG PAP within sub-measurement, r is the index of CPMG within the pair (e.g., it spans just two indices denoting positive or negative CPMG), the n-th (n is again the echo index within CPMG n=1, . . . NEₖ) spin echo amplitude for a fluid of relative volume P with single T1 and T2 relaxation times is given by the equation:

$${E}_{k , l , r , n} = {C}_{k , l , r} \cdot P [ 1 - exp ( - \frac{{WT}_{k}}{T 1} ) ] exp ( - \frac{n \cdot {TE}_{k}}{T 2} ) + {w}_{n} \qquad ( 3 )$$

where coefficient C_{k,l,s} either ½ for a positive CPMG of a PAP or −½ for a negative CPMG. Equation (3) again assumes uniform and static magnetic field and infinite antenna Q.

As a rule, PAPS-ing is performed. That is, negative CPMG echoes of a PAP are subtracted from positive and upon that results for all CPMG of the sub-measurement are summed together with the sum divided to the number of CPMG pairs. Upon PAPS-ing the spin echo amplitude is governed by the equation:

$${E}_{k , n} = P [ 1 - exp ( - \frac{{WT}_{k}}{T 1} ) ] exp ( - \frac{n \cdot {TE}_{k}}{T 2} ) + {\tilde{w}}_{k , n} \qquad ( 4 )$$

Noise \tilde{w}_{k,n} expressed in the equation (4) is one specific to the k-th sub-measurement. \tilde{w}_{k,n} designates noise realizations for the k-th sub-measurement, with noise magnitude denoted as |\tilde{w}|ₖ. Commonly, CPMGs with shorter WT and less NE designed to gather information on T1 employ more PAPs and, therefore, noise magnitude for them is reduced by PAPS-ing. Consequently, noise magnitude is estimated on per sub-measurement basis.

In cases when NMR depth logging is performed, reduction of noise can be done by summing several echo trains obtained at the adjacent depth and dividing the sum to the number of depths. This procedure is called stacking. In addition, parts of the acquisition sequence can be recorded at the adjacent depths in order to speed logging. Typically, one set of the longest first CPMG and half CPMG pairs of all other sub-measurements is acquired, and then at the next depth phase-alternate set of the longest CPMG with another half of CPMG pairs is acquired. Sequences acquired at adjacent depth is then merged, PAPS-ed and stacked. The embodiments described herein do not involve modification T1-T2 sequence acquisition techniques and PAPS-ing including relevant modifications of the phase-rotation, noise evaluation, antenna gain correction, temperature correction and calibration algorithms. Rather, the embodiments described herein assume that the algorithms established in the industry are used.

Thus, the spin echoes for a fluid with single T1 and T2 relaxation times with uniform and static magnetic field and infinite antenna Q is governed by the equation (4). Various inversion techniques such as Tikhonov regularization, rigorous Bayesian inference methods, and heuristic stochastics methods use that relationship to deliver the fluid volume distribution P(T1, T2) with respect to the relaxation times that corresponds to the observed echoes amplitudes E_{k,n} and sub-measurement noise of the magnitude Wk. The multiplicative term that renders echoes amplitudes (or their window sums) from the fluid volume for specific relaxation times P (T1,T2) is called NMR kernel. The NMR kernel for individual echoes corresponding to the equation (4) is:

$${K}_{k , n} ( T 1 , T 2 ) = [ 1 - exp ( - \frac{{WT}_{k}}{T 1} ) ] exp ( - \frac{n \cdot {TE}_{k}}{T 2} ) \qquad ( 5 )$$

The embodiments described herein include various methods of applying a cutoff, gaussian distribution, or differential gaussian distribution in the T1 domain, T2 domain, or two-dimensional T1-T2 domain to an NMR log acquired in a well to predict various parameters relating to CO₂ in well fluids. It will be appreciated that the calculations and predictions that are described in greater detail herein may be performed by the data processing system 38 described herein.

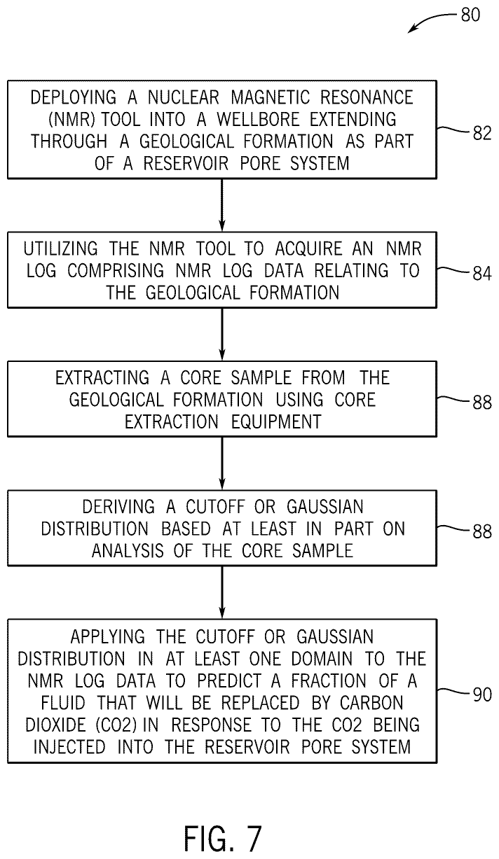

For example, FIG. 7 illustrates a method 80 that includes deploying an NMR tool 12 into a wellbore 16 extending through a geological formation 14 as part of a reservoir pore system 10 (step 82). The method 80 also includes utilizing the NMR tool 12 to acquire an NMR log that includes NMR log data relating to the geological formation 14 (step 84). The method 80 further includes extracting a core sample from the geological formation 14 using core extraction equipment (e.g., a wireline rotary sidewall coring tool) (step 86). It should be noted that step 86 may be performed before or after steps 82 and 84, and may equally well be performed while drilling the well using coring equipment that forms part of a drilling bottomhole assembly. The method 80 also includes deriving a cutoff or gaussian distribution based at least in part on analysis of the core sample (step 88). The method 80 further includes applying the cutoff or gaussian distribution in at least one domain to the NMR log data to predict a fraction of a fluid that will be replaced by CO₂ in response to the CO₂ being injected into the reservoir pore system 10 (step 90).

As described in greater detail herein, in certain embodiments, deriving the cutoff or gaussian distribution may be performed by introducing a known amount of CO₂ into the core sample in laboratory conditions, and measuring it before, after (and during) the injection of CO₂ with a laboratory NMR setup that resembles that of the NMR tool 12 as closely as possible. This analysis is how the gaussians and/or the cutoffs to be applied to the NMR data from the downhole tool may be determined. This applies to any or all of the other deriving steps described herein.

In certain embodiments, the method 80 includes modifying well testing operations and a sequence of injected fluids and measurements based at least in part on the predicted fraction of the fluid. For example, in certain embodiments, control signals may be transmitted (e.g., from the data processing system 28) to a downhole well tool disposed in a wellbore of the reservoir pore system 10 to automatically adjust injection parameters (e.g., flow rates, pressures, and so forth) of fluids such as CO₂ being injected into the reservoir pore system 10 in accordance with the predicted fraction of the fluid by, for example, controlling mechanical settings of equipment (e.g., such as valves, pumps, and so forth) of the downhole well tool. In certain embodiments, the at least one domain includes the T2 domain. In other embodiments, the at least one domain includes the T1 domain. In yet other embodiments, the at least one domain includes the two-dimensional T1-T2 domain.

In certain embodiments, the method 80 includes deriving the cutoff or gaussian distribution from differences observed between NMR spectra before and after centrifuging of the core sample in air or in a specifically chosen fluid at ambient temperature and pressure or at another specifically chosen temperature and pressure. In certain embodiments, the method 80 includes deriving the cutoff or gaussian distribution from differences observed between NMR spectra before, after, or while instigating changes in CO₂ saturation during analysis of the core sample using core analysis equipment 48. In such embodiments, the method 80 may include conducting the analysis of the core sample at reservoir pressure and temperature or at another specifically chosen pressure and temperature. In certain embodiments, the method 80 includes deriving the cutoff or gaussian distribution from differences observed between NMR spectra before and after immersing one end of the core sample in CO₂ while applying a vacuum to the other end of the core sample. In certain embodiments, the method 80 includes determining the cutoff or gaussian distribution by history matching downhole measurements of saturation following injection of CO₂ into the geological formation 14.

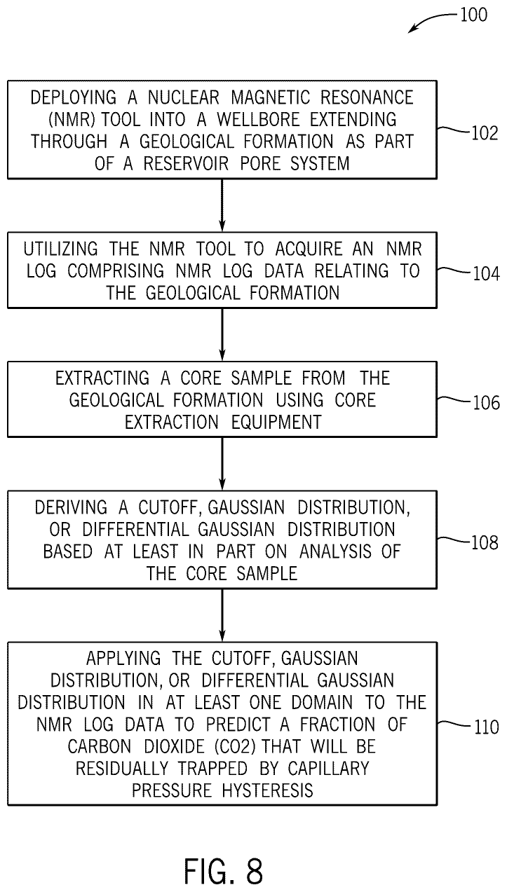

FIG. 8 illustrates another method 100 that includes deploying an NMR tool 12 into a wellbore 16 extending through a geological formation 14 as part of a reservoir pore system 10 (step 102). The method 100 also includes utilizing the NMR tool 12 to acquire an NMR log that includes NMR log data relating to the geological formation 14 (step 104). The method 100 further includes extracting a core sample from the geological formation 14 using core extraction equipment (e.g., a wireline rotary sidewall coring tool) (step 106). It should be noted that step 106 may be performed before or after steps 102 and 104, and may equally well be performed while drilling the well using coring equipment that forms part of a drilling bottomhole assembly. The method 100 also includes deriving a cutoff, gaussian distribution, or differential gaussian distribution based at least in part on analysis of the core sample (step 108). The method 100 further includes applying the cutoff, gaussian distribution, or differential gaussian distribution in at least one domain to the NMR log data to predict a fraction of CO₂ that will be residually trapped by capillary pressure hysteresis (step 110).

In addition, in certain embodiments, the method 100 includes modifying well testing operations and a sequence of injected fluids and measurements based at least in part on the predicted fraction of CO₂. For example, in certain embodiments, control signals may be transmitted (e.g., from the data processing system 28) to a downhole well tool disposed in a wellbore of the reservoir pore system 10 to automatically adjust injection parameters (e.g., flow rates, pressures, and so forth) of fluids such as CO₂ being injected into the reservoir pore system 10 in accordance with the predicted fraction of CO₂ by, for example, controlling mechanical settings of equipment (e.g., such as valves, pumps, and so forth) of the downhole well tool.

In certain embodiments, the at least one domain includes the T2 domain. In other embodiments, the at least one domain includes the T1 domain. In yet other embodiments, the at least one domain includes the two-dimensional T1-T2 domain. In certain embodiments, the method 100 includes deriving the cutoff, gaussian distribution, or differential gaussian distribution from differences observed between NMR spectra before, after, or while instigating changes in CO₂ saturation during analysis of the core sample using core analysis equipment 48. In such embodiments, the method 100 may include conducting the analysis of the core sample at reservoir pressure and temperature or at another specifically chosen pressure and temperature. In certain embodiments, the method 100 includes determining the cutoff, gaussian distribution, or differential gaussian distribution by history matching downhole measurements of saturation following injection of CO₂ and/or water into the geological formation 14.

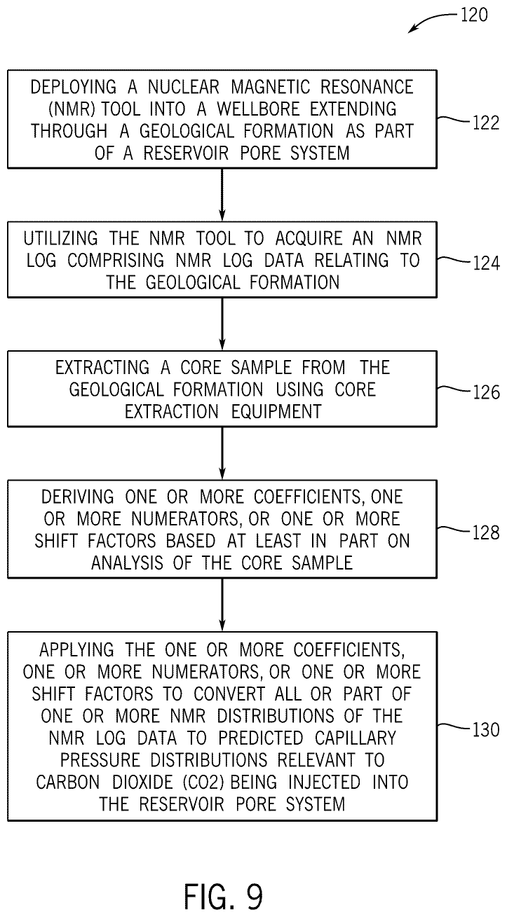

FIG. 9 illustrates another method 120 that includes deploying an NMR tool 12 into a wellbore 16 extending through a geological formation 14 as part of a reservoir pore system 10 (step 122). The method 120 also includes utilizing the NMR tool 12 to acquire an NMR log that includes NMR log data relating to the geological formation 14 (step 124). The method 120 further includes extracting a core sample from the geological formation 14 using core extraction equipment (e.g., a wireline rotary sidewall coring tool) (step 126). It should be noted that step 126 may be performed before or after steps 122 and 124, and may equally well be performed while drilling the well using coring equipment that forms part of a drilling bottomhole assembly. The method 120 also includes deriving one or more coefficients, one or more numerators, or one or more shift factors based at least in part on analysis of the core sample (step 128). The method 120 further includes applying the one or more coefficients, one or more numerators, or one or more shift factors to convert all or part of one or more NMR distributions of the NMR log data to predicted capillary pressure distributions relevant to CO₂ being injected into the reservoir pore system 10 (step 130).

In addition, in certain embodiments, the method 120 includes modifying well testing operations and/or a sequence of injected fluids and measurements based at least in part on the predicted capillary pressure distributions. For example, in certain embodiments, control signals may be transmitted (e.g., from the data processing system 28) to a downhole well tool disposed in a wellbore of the reservoir pore system 10 to automatically adjust injection parameters (e.g., flow rates, pressures, and so forth) of fluids such as CO₂ being injected into the reservoir pore system 10 in accordance with the predicted capillary pressure distributions by, for example, controlling mechanical settings of equipment (e.g., such as valves, pumps, and so forth) of the downhole well tool.

In certain embodiments, the one or more NMR distributions include T2 distributions. In other embodiments, the one or more NMR distributions include T1 distributions. In yet other embodiments, the one or more NMR distributions include two-dimensional T1-T2 maps. In certain embodiments, the method 120 includes determining the one or more coefficients, one or more numerators, or one or more shift factors by comparing NMR T2 data acquired at multiple steps of CO₂ relative pressure and/or saturation induced in a laboratory capillary pressure experiment of the core sample. In such embodiments, the method 120 may include performing the laboratory capillary pressure experiment of the core sample at reservoir temperature and pressure or at other specifically designed pressure and temperature conditions.

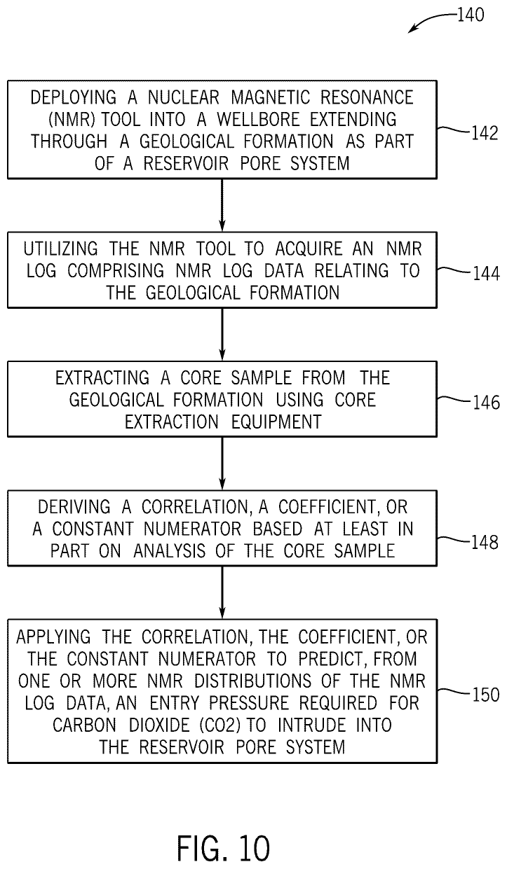

FIG. 10 illustrates another method 140 that includes deploying an NMR tool 12 into a wellbore 16 extending through a geological formation 14 as part of a reservoir pore system 10 (step 142). The method 140 also includes utilizing the NMR tool 12 to acquire an NMR log that includes NMR log data relating to the geological formation 14 (step 144). The method 140 further includes extracting a core sample from the geological formation 14 using core extraction equipment (e.g., a wireline rotary sidewall coring tool) (step 146). It should be noted that step 146 may be performed before or after steps 142 and 144, and may equally well be performed while drilling the well using coring equipment that forms part of a drilling bottomhole assembly. The method 140 also includes deriving a correlation, a coefficient, or a constant numerator based at least in part on analysis of the core sample (step 148). The method 140 further includes applying the correlation, the coefficient, or the constant numerator to predict, from one or more NMR distributions of the NMR log data, an entry pressure required for CO₂ to intrude into the reservoir pore system 10 (step 150).

In addition, in certain embodiments, the method 140 includes modifying well testing operations and a sequence of injected fluids and measurements based at least in part on the predicted entry pressure. For example, in certain embodiments, control signals may be transmitted (e.g., from the data processing system 28) to a downhole well tool disposed in a wellbore of the reservoir pore system 10 to automatically adjust injection parameters (e.g., flow rates, pressures, and so forth) of fluids such as CO₂ being injected into the reservoir pore system 10 in accordance with the predicted entry pressure by, for example, controlling mechanical settings of equipment (e.g., such as valves, pumps, and so forth) of the downhole well tool.

In certain embodiments, the one or more NMR distributions include a T2 distribution. In other embodiments, the one or more NMR distributions include a T1 distribution. In yet other embodiments, the one or more NMR distributions include a two-dimensional T1-T2 map. In certain embodiments, the method 140 includes determining the correlation, the coefficient, or the constant numerator by comparing relative injection pressure and NMR data before injection of CO₂ into the core sample and/or during ramping of CO₂ relative pressure and/or after CO₂ has reached a target level of saturation in the core sample during analysis of the core sample. In such embodiments, the method 140 may include conducting the analysis of the core sample at reservoir pressure and temperature or at another specifically chosen pressure and temperature.

For the methods 80, 100, 120, 140 illustrated in FIGS. 7-10, instead of pure CO₂, analysis may be carried out based on an impure mixture of CO₂ and other substances, for example, including but not limited to air, water, nitrogen, methane, hydrogen sulfide, nitrous oxide, ammonia, and so forth. In other words, in certain embodiments, testing may be performed in a laboratory with fluids that are different from the fluids that will be injected into the reservoir pore system 10. In addition, for the methods 80, 100, 120, 140 illustrated in FIGS. 7-10, fluid substitution corrections may first be applied to the T1 and/or T2 distributions to be interpreted to correct for the presence of oil or other non-aqueous fluids and produce a fully water-saturated equivalent T1 and/or T2 distribution. The purpose of fluid substitution corrections is to take a T2 distribution from a formation (e.g., log or lab measurement) where both water and oil are present, and generate a corrected T2 distribution as if water were the only fluid in the pores. This is especially useful for wells drilled with oil-based drilling fluids, where oil filtrate may have invaded the pore system 10 displacing water as part of the drilling process.

In addition, for the methods 80, 100, 120, 140 illustrated in FIGS. 7-10, the water-saturated equivalent distribution may be an input for the interpretation and the analysis. In addition, for the methods 80, 100, 120, 140 illustrated in FIGS. 7-10, the fluid substitution correction may be determined using actual laboratory measurements on core samples before and after fluid substitution. In addition, for the methods 80, 100, 120, 140 illustrated in FIGS. 7-10, based on knowledge, measurement, or assumption of formation mineralogy and/or paramagnetic and magnetic mineral content, T1 and T2 distributions may be corrected prior to interpretation. In addition, for the methods 80, 100, 120, 140 illustrated in FIGS. 7-10, the coefficients, constants, or correlations may be adjusted for the effect of mineralogy.

In addition, for the methods 80, 100, 120, 140 illustrated in FIGS. 7-10, to produce a predicted continuous profile, vertically, along the wellbore trajectory, or in a direction of true stratigraphic thickness, of related formation properties including but not limited to injectate (CO₂) relative permeability, injectate (CO₂) maximum saturation, injectate (CO₂)-brine displacement efficiency, injectate (CO₂)-brine capillary pressure vs. saturation profile, injectate (CO₂) threshold entry pressure. In addition, for the methods 80, 100, 120, 140 illustrated in FIGS. 7-10, the continuous property profiles may be determined to delineate flow units and baffles or barriers to flow of injectate (CO₂) at original, nominal, or varied levels of reservoir pressure and injection pressure. In addition, the methods 80, 100, 120, 140 illustrated in FIGS. 7-10 may include estimation of vertical effective and relative permeability (or vertical/horizontal permeability ratio) to injectate (CO₂).

In addition, the methods 80, 100, 120, 140 illustrated in FIGS. 7-10 may include populating static or dynamic reservoir models with properties with an aim to improving the representativeness of such properties, representation of the true statistical variation of such properties, and/or the predictive accuracy of the simulation outputs. Simulation outputs targeted for improved accuracy may include, but are not limited to, (a) three dimensional distribution and velocity of the CO₂ plume and CO₂ saturation vs. time, (b) arrival time of the CO₂ plume at specific landmarks, (c) distribution and magnitude of the pressure plume, (d) accumulation rate and overpressure of continuous CO₂ columns structurally trapped beneath confining formations, (e) percentage or mass of CO₂ vs. time becoming residually trapped, (f) percentage or mass of CO₂ vs. time entering aqueous solution, and so forth.

As such, FIGS. 7-10 illustrate various methods of combining NMR log data with core sample analysis to determine various parameters relating to CO₂ to be injected into the reservoir pore system 10. For example, a first method may include applying a cutoff or gaussian distribution, however measured, derived, or conceived, in the T2 domain to an NMR log acquired in a well (e.g., the reservoir pore system 10 illustrated in FIG. 1), thereby predicting a fraction of a fluid that will be replaced by CO₂ in response to the CO₂ being introduced to a system comprising a plurality of fluids, the plurality of fluids including at least water or brine. For example, the cutoff or gaussian distribution may be derived from differences observed between NMR spectra before and after centrifuging of the core sample either in air or in a specifically chosen fluid at ambient temperature of pressure or at another specifically chosen temperature and pressure. In addition, the cutoff or gaussian distribution may be derived from differences observed between NMR spectra before, after, or while instigating changes in CO₂ saturation using core flooding equipment, relative permeability equipment, or other special core analysis equipment 48. In addition, the method may be conducted at reservoir pressure and temperature or at another specifically chosen pressure and temperature. In addition, the cutoff or gaussian distribution may be derived from differences observed between NMR spectra before and after immersing one end of the sample in CO₂ while applying a vacuum to the other end of the sample.

Another method may include applying a cutoff or gaussian distribution, however measured, derived, or conceived, in the T1 domain to an NMR log acquired in a well (e.g., the reservoir pore system 10 illustrated in FIG. 1), thereby predicting a fraction of a fluid that will be replaced by CO₂ in response to the CO₂ being introduced to a system comprising a plurality of fluids, the plurality of fluids including at least water or brine. For example, the cutoff or gaussian distribution may be derived from differences observed between NMR spectra before and after centrifuging of the core sample in air or in a specifically chosen fluid at ambient temperature or pressure or at another specifically chosen temperature and pressure. In addition, the cutoff or gaussian distribution may be derived from differences observed between NMR spectra before, after, or while instigating changes in CO₂ saturation using core flooding equipment, relative permeability equipment, or other special core analysis equipment 48. In addition, the method may be conducted at reservoir pressure and temperature or at another specifically chosen pressure and temperature. In addition, the cutoff or gaussian distribution may be derived from differences observed between NMR spectra before and after immersing one end of the sample in CO₂ while applying a vacuum to the other end of the sample.

Another method may include applying a two-dimensional (2D) cutoff or 2D gaussian distribution, however measured, derived, or conceived, in the two-dimensional T1-T2 domain to an NMR log acquired in a well (e.g., the reservoir pore system 10 illustrated in FIG. 1), thereby predicting a fraction of a fluid that will be replaced by CO₂ in response to the CO₂ being introduced to a system comprising a plurality of fluids, the plurality of fluids including at least water or brine. For example, the cutoff or gaussian distribution may be derived from differences observed between NMR spectra before and after centrifuging of the core sample in air or in a specifically chosen fluid at ambient temperature and pressure or at another specifically chosen temperature and pressure. In addition, the cutoff or gaussian distribution may be derived from differences observed between NMR spectra before, after, or while instigating changes in CO₂ saturation using core flooding equipment, relative permeability equipment, or other special core analysis equipment 48. In addition, the method may be conducted at reservoir pressure and temperature or at another specifically chosen pressure and temperature. In addition, the cutoff or gaussian distribution may be derived from differences observed between NMR spectra before and after immersing one end of the sample in CO₂ while applying a vacuum to the other end of the sample.

Another method may include applying a cutoff, gaussian distribution, or differential gaussian distribution in combination, with or without a trapping coefficient, however measured, derived, or conceived, in the T₂ domain to an NMR log acquired in a well (e.g., the reservoir pore system 10 illustrated in FIG. 1) to predict the fraction of CO₂ that will be residually trapped by capillary pressure hysteresis. For example, the cutoff, gaussian distribution, differential gaussian distribution, and/or trapping coefficient may be derived from differences observed between NMR spectra before, after, or while instigating changes in CO₂ saturation using core flooding equipment, relative permeability equipment, or other special core analysis equipment 48. In addition, the method may be conducted at reservoir pressure and temperature or at another specifically chosen pressure and temperature.

Another method may include applying a cutoff, gaussian distribution, or differential gaussian distribution in combination, with or without a trapping coefficient, however measured, derived, or conceived, in the T1 domain to an NMR log acquired in a well (e.g., the reservoir pore system 10 illustrated in FIG. 1) to predict the fraction of CO₂ that will be residually trapped by capillary pressure hysteresis. For example, the cutoff, gaussian distribution, differential gaussian distribution, and/or trapping coefficient may be derived from differences observed between NMR spectra before, after, or while instigating changes in CO₂ saturation using core flooding equipment, relative permeability equipment, or other special core analysis equipment 48. In addition, the method may be conducted at reservoir pressure and temperature or at another specifically chosen pressure and temperature.

Another method may include applying a cutoff, gaussian distribution, or differential gaussian distribution in combination, with or without a trapping coefficient, however measured, derived, or conceived, in the two-dimensional T1-T2 domain to an NMR log acquired in a well (e.g., the reservoir pore system 10 illustrated in FIG. 1) to predict the fraction of CO₂ that will be residually trapped by capillary pressure hysteresis. For example, the cutoff, gaussian distribution, differential gaussian distribution, and/or trapping coefficient is derived from differences observed between NMR spectra before, after, or while instigating changes in CO₂ saturation using core flooding equipment, relative permeability equipment, or other special core analysis equipment 48. In addition, the method may be conducted at reservoir pressure and temperature or at another specifically chosen pressure and temperature.

Another method may include applying a coefficient or coefficients, numerator or numerators, shift factor or factors, however measured, derived, or conceived, to convert all or part of NMR T2 distributions to predicted capillary pressure distributions relevant to CO₂ intruding into a system (e.g., the reservoir pore system 10 illustrated in FIG. 1) of one or more fluids including water or brine. For example, the derivation of coefficients, constant numerators, or shift factors may be determined by comparing NMR T2 data acquired at multiple steps of CO₂ relative pressure and/or saturation induced in a laboratory capillary pressure experiment of the core sample. In addition, the laboratory capillary pressure experiment of the core sample may be performed at reservoir temperature and pressure, or at other specifically designed pressure and temperature conditions.

Another method may include applying a coefficient or coefficients, numerator or numerators, shift factor or factors, however measured, derived, or conceived, to convert all or part of NMR T1 distributions to predicted capillary pressure distributions relevant to CO₂ intruding into a system (e.g., the reservoir pore system 10 illustrated in FIG. 1) of one or more fluids including water or brine. For example, the derivation of coefficients, constant numerators, or shift factors may be determined by comparing NMR T2 data acquired at multiple steps of CO₂ relative pressure and/or saturation induced in a laboratory capillary pressure experiment of the core sample. In addition, the laboratory capillary pressure experiment of the core sample may be performed at reservoir temperature and pressure, or other specifically designed pressure and temperature conditions.

Another method may include applying a coefficient or coefficients, numerator or numerators, shift factor or factors, however measured, derived, or conceived, to convert all or part of two dimensional NMR T1-T2 maps to predicted capillary pressure distributions relevant to CO₂ intruding into a system (e.g., the reservoir pore system 10 illustrated in FIG. 1) of one or more fluids including water or brine. For example, the derivation of coefficients, constant numerators, or shift factors may be determined by comparing NMR T2 data acquired at multiple steps of CO₂ relative pressure and/or saturation induced in a laboratory capillary pressure experiment of the core sample. In addition, the laboratory capillary pressure experiment of the core sample may be performed at reservoir temperature and pressure, or other specifically designed pressure and temperature conditions.

Another method may include applying a correlation, a coefficient, or a constant numerator, however measured, derived, or conceived, to predict from a T2 distribution or part thereof an entry pressure required for CO₂ to intrude into the pore system of a rock containing one or more fluid phases including brine or water. For example, the correlations, coefficients, or constants may be determined by comparing relative injection pressure and NMR data before injection of CO₂ into a laboratory sample, and/or during ramping of CO₂ relative pressure, and/or after CO₂ has reached a target level of saturation (e.g. 10%) in the pore system of the sample. In addition, the laboratory experiments may be carried out at reservoir temperature and pressure or at another specifically designed temperature and pressure.

Another method may include applying a correlation, a coefficient, or a constant numerator, however measured, derived, or conceived, to predict from a T1 distribution or part thereof an entry pressure required for CO₂ to intrude into the pore system of a rock containing one or more fluid phases including brine or water. For example, the correlations, coefficients, or constants may be determined by comparing relative injection pressure and NMR data before injection of CO₂ into a laboratory sample, and/or during ramping of CO₂ relative pressure, and/or after CO₂ has reached a target level of saturation (e.g. 10%) in the pore system of the sample. In addition, the laboratory experiments may be carried out at reservoir temperature and pressure or at another specifically designed temperature and pressure.

Another method may include applying a correlation, a coefficient, or a constant numerator, however measured, derived, or conceived, to predict from a T1-T2 map or part thereof an entry pressure required for CO₂ to intrude into the pore system of a rock containing one or more fluid phases including brine or water. For example, the correlations, coefficients, or constants may be determined by comparing relative injection pressure and NMR data before injection of CO₂ into a laboratory sample, and/or during ramping of CO₂ relative pressure, and/or after CO₂ has reached a target level of saturation (e.g. 10%) in the pore system of the sample. In addition, the laboratory experiments may be carried out at reservoir temperature and pressure or at another specifically designed temperature and pressure.

For all of these methods, instead of pure CO₂, analysis may be carried out based on an impure mixture of CO₂ and other substances, for example, including but not limited to air, water, nitrogen, methane, hydrogen sulfide, nitrous oxide, ammonia, and so forth. In addition, for all of these methods, the fluid substitution corrections, however determined or derived, may first be applied to the T1 and/or T2 distributions to be interpreted to correct for the presence of oil or other non-aqueous fluids and produce a fully water-saturated equivalent T1 and/or T2 distribution. In addition, for all of these methods, the water-saturated equivalent distribution may be an input for the interpretation and the analysis. In addition, for all of these methods, the fluid substitution correction may be determined using actual laboratory measurements on core samples before and after fluid substitution. In addition, for all of these methods, based on knowledge, measurement, or assumption of formation mineralogy and/or paramagnetic and magnetic mineral content, T1 and T2 distributions may be corrected prior to interpretation. In addition, for all of these methods, the coefficients, constants, or correlations may be adjusted for the effect of mineralogy.

In addition, for all of these methods, to produce a predicted continuous profile, vertically, along the wellbore trajectory, or in a direction of true stratigraphic thickness, of related formation properties including but not limited to injectate (CO₂) relative permeability, injectate (CO₂) maximum saturation, injectate (CO₂)-brine displacement efficiency, injectate (CO₂)-brine capillary pressure vs. saturation profile, injectate (CO₂) threshold entry pressure. In addition, for all of these methods, the continuous property profiles may be determined to delineate flow units and baffles or barriers to flow of injectate (CO₂) at original, nominal, or varied levels of reservoir pressure and injection pressure. In addition, all of these methods may include estimation of vertical effective and relative permeability (or vertical/horizontal permeability ratio) to injectate (CO₂).

In addition, all of these methods may include populating static or dynamic reservoir models with properties with an aim to improving the representativeness of such properties, representation of the true statistical variation of such properties, and/or the predictive accuracy of the simulation outputs. Simulation outputs targeted for improved accuracy may include, but are not limited to, (a) three dimensional distribution and velocity of the CO₂ plume and CO₂ saturation vs. time, (b) arrival time of the CO₂ plume at specific landmarks, (c) distribution and magnitude of the pressure plume, (d) accumulation rate and overpressure of continuous CO₂ columns structurally trapped beneath confining formations, (e) percentage or mass of CO₂ vs. time becoming residually trapped, (f) percentage or mass of CO₂ vs. time entering aqueous solution, and so forth.

Another method of deriving and applying a cutoff or gaussian distribution, as described in greater detail herein, may include one of two example scenarios. The first example scenario is where the operator makes a decision to develop the project, and eventually commences injection operations. After a period of CO₂ injection, logging measurements may be measured in injection or monitoring wells to measure actual reservoir CO₂ saturation. The NMR logs from months or years earlier, when the project was first evaluated may then be reinterpreted (e.g., the cutoffs or gaussians may be “calibrated”) to history match the new measurements. The second example scenario is where, as part of well evaluation, a wireline formation tester may be used to inject a small quantity of CO₂. Various logs acquired before and after injection may be used to quantify the change in saturation, then the NMR interpretation parameters may be calibrated to give the correct prediction based on pre-injection data. Both of these example scenarios may be referred to as “history matching”.

The tangible and non-transitory machine-readable medium of any preceding clause, wherein the instructions further cause the processing system to determine whether to utilize the NMR logging measurements and core sample analysis in generation of estimations of formation attributes.

This written description uses examples to disclose the subject matter, including the best mode, and also to enable any person skilled in the art to practice the subject matter, including making and using any devices or systems and performing any incorporated methods. The patentable scope of the subject matter is defined by the claims and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The techniques presented and claimed herein are referenced and applied to material objects and concrete examples of a practical nature that demonstrably improve the present technical field and, as such, are not abstract, intangible, or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112 (f). However, for any claims containing elements designated in any other manner, it is intended that such elements are not to be interpreted under 35 U.S.C. § 112 (f).

What is claimed is:

1. A method comprising:
deploying a Nuclear Magnetic Resonance (NMR) tool into a wellbore extending through a geological formation as part of a reservoir pore system;
utilizing the NMR tool to acquire an NMR log comprising NMR log data relating to the geological formation extracting a core sample from the geological formation using core extraction equipment;
deriving a cutoff or gaussian distribution based at least in part on analysis of the core sample; and
applying the cutoff or gaussian distribution in at least one domain to the NMR log data to predict a fraction of a fluid that will be replaced by carbon dioxide (CO₂) in response to the CO₂ being injected into the reservoir pore system.

2. The method of claim 1, wherein the at least one domain comprises the T2 domain.

3. The method of claim 1, wherein the at least one domain comprises the T1 domain.

4. The method of claim 1, wherein the at least one domain comprises the two-dimensional T1-T2 domain.

5. The method of claim 1, comprising deriving the cutoff or gaussian distribution from differences observed between NMR spectra before and after centrifuging of the core sample in air or in a specifically chosen fluid at ambient temperature and pressure or at another specifically chosen temperature and pressure.

6. The method of claim 1, comprising deriving the cutoff or gaussian distribution from differences observed between NMR spectra before, after, or while instigating changes in CO₂ saturation during analysis of the core sample using core analysis equipment.

7. The method of claim 6, comprising conducting the analysis of the core sample at reservoir pressure and temperature or at another specifically chosen pressure and temperature.

8. The method of claim 1, comprising deriving the cutoff or gaussian distribution from differences observed between NMR spectra before and after immersing one end of the core sample in CO₂ while applying a vacuum to the other end of the core sample.

9. The method of claim 1, comprising determining the cutoff or gaussian distribution by history matching downhole measurements of saturation following injection of CO₂ into the geological formation.

10. A method comprising:
deploying a Nuclear Magnetic Resonance (NMR) tool into a wellbore extending through a geological formation as part of a reservoir pore system;
utilizing the NMR tool to acquire an NMR log comprising NMR log data relating to the geological formation;
extracting a core sample from the geological formation using core extraction equipment;
deriving a cutoff, gaussian distribution, or differential gaussian distribution based at least in part on analysis of the core sample; and
applying the cutoff, gaussian distribution, or differential gaussian distribution in at least one domain to the NMR log data to predict a fraction of carbon dioxide (CO₂) that will be residually trapped by capillary pressure hysteresis.

11. The method of claim 10, wherein the at least one domain comprises the T2 domain.

12. The method of claim 10, wherein the at least one domain comprises the T1 domain.

13. The method of claim 10, wherein the at least one domain comprises the two-dimensional T1-T2 domain.

14. The method of claim 10, comprising deriving the cutoff, gaussian distribution, or differential gaussian distribution from differences observed between NMR spectra before, after, or while instigating changes in CO₂ saturation during analysis of the core sample using core analysis equipment.

15. The method of claim 14, comprising conducting the analysis of the core sample at reservoir pressure and temperature or at another specifically chosen pressure and temperature.

16. The method of claim 10, comprising determining the cutoff, gaussian distribution, or differential gaussian distribution by history matching downhole measurements of saturation following injection of CO₂ and/or water into the geological formation.

17. A method comprising:
deploying a Nuclear Magnetic Resonance (NMR) tool into a wellbore extending through a geological formation as part of a reservoir pore system;
utilizing the NMR tool to acquire an NMR log comprising NMR log data relating to the geological formation;
extracting a core sample from the geological formation using core extraction equipment;
deriving one or more coefficients, one or more numerators, or one or more shift factors based at least in part on analysis of the core sample; and
applying the one or more coefficients, one or more numerators, or one or more shift factors to convert all or part of one or more NMR distributions of the NMR log data to predicted capillary pressure distributions relevant to carbon dioxide (CO₂) being injected into the reservoir pore system.

18. The method of claim 17, wherein the one or more NMR distributions comprise T2 distributions.

19. The method of claim 17, wherein the one or more NMR distributions comprise T1 distributions.

20. The method of claim 17, wherein the one or more NMR distributions comprise two-dimensional T1-T2 maps.

21. The method of claim 17, comprising determining the one or more coefficients, one or more numerators, or one or more shift factors by comparing NMR T2 data acquired at multiple steps of CO₂ relative pressure and/or saturation induced in a laboratory capillary pressure experiment of the core sample.

22. The method of claim 21, comprising performing the laboratory capillary pressure experiment of the core sample at reservoir temperature and pressure or at other specifically designed pressure and temperature conditions.
